(19) United States
(12) Patent Application Publication
(10) Pub. No.: US 2012/0118819 A1
(43) Pub. Date: May 17, 2012

(54) IN-SITU, MICROBIAL BIO-REMEDIATION OF AQUATIC ENVIRONMENTS

(76) Inventor: Israel Amichay Bachar, Petach Tiqwa (IL)

(21) Appl. No.: 12/943,957

(22) Filed: Nov. 11, 2010

(51) Int. Cl. C02F 3/10; C12N 1/00; B09C 1/00

(52) U.S. Cl. 210/616; 210/615; 210/619; 435/243

(57) ABSTRACT

A method for facilitating growth of microbial communities for in-situ bodies of flowing water for the sake of bio-remediating contaminated water and/or producing useful biomass.

### FIELD AND BACKGROUND OF THE INVENTION

The present invention relates to water pollution and, in particular, it is concerned with removing or neutralizing environmental pollutants from bodies of flowing water such as rivers, streams, brooks, creeks, lakes, ponds, coastlines via naturally grown, complex-microbial-communities on artificial surfaces deployed in these habitats.

As is well-known, water pollution has immense ecological, agricultural and financial significance. Many water bodies around the world, polluted primarily from industrial, agricultural, and domestic wastes, are so polluted that they lack any commercial or recreational value, constitute a health hazards, and lack the ability to sustain multicellular life.

Most water treatment technologies focus on wastewater treatment or treatment of standing waters before being discarded into the environment. The drawback of these technologies is that they require special pumping equipment and storage facilities to convey and to store the polluted water during treatment. Besides the additional capital and operational costs, underdeveloped societies lack technological infrastructures are unable to benefit from such systems.

Some of natural water treatment occurs by way of complex microbial mats formed in aquatic environments. Biofilms and photosynthetic Cyanobacterial mats always form on illuminated submerged surfaces in aquatic environments, regardless of the composition of the submerged surface. The biological diversity of such natural mats is immense and includes populations of living cells adapted to their specific environment that can absorb, restore and exploit pollutants for their own growth. In heavily polluted environments naturally occurring biofilms or microbial mats develop in quantities insufficient to bioremediate the pollution. This microbial shortage is further compounded by multi-cellular organisms constantly feeding on them.

The present invention addresses this problem by introducing artificial surfaces into the polluted water to increase the microbial biomass per cubic meter of flowing water to an extent needed to effectively bioremediate the pollution.

U.S. Pat. No. 6,033,559 teaches bio-remediation by way of constructed microbial mat into an aquatic environment; however, in an effort to immobilize the microbial community, the mats are cultured inside a glass wool mesh and then introduced into aqueous environments in. Such arrangements are limited to bio-remediating bodies of water of a fixed volume in which the pollute water contacts the microbial mats for an extended period of time or in situations in which the polluted water repeatedly contacts the mats for short period of time. Pre-cultured biomats are therefore ineffective for bio-remediating flowing bodies of water in which the polluted water contacts the mats only during a single pass over the mats.

Therefore, there is a need for system facilitating microbial growth in-situ bodies of flowing water to facilitate bioremediation in those environments.

### SUMMARY OF THE INVENTION

The present invention is a method for facilitating growth of microbial communities for in-situ bodies of flowing water for the sake of bio-remediating contaminated water and/or producing useful biomass.

According to the teachings of the present invention there is provided, a method for growing microbial communities in flowing water, which comprises inserting at least a portion of at least one sheet of material into an in-situ body of water flowing downstream; and holding the sheet in the in-situ body of water flowing downstream by way of a sheet deployment structure configured to hold the sheet so as to maximize contact of the body of water flowing downstream with surfaces of the sheets, thereby facilitating bio-remediation of the body of water by way of microbial colonization on the surfaces of the at least one sheet.

According to a further feature of the present invention, the sheet of material includes a screen.

According to a further feature of the present invention, the sheet of material includes a polymeric material.

According to a further feature of the present invention, the inserting at least one sheet of material includes orientating the sheet in a substantially non-vertical plane.

According to a further feature of the present invention, the inserting at least one sheet of material includes orientating the sheet of material in a substantially vertical plane.

According to a further feature of the present invention, the inserting at least one sheet of material includes inserting each of at least one of the sheets into the in-situ body of water by unwinding each of the at least one sheet from a corresponding rolled sheet of material held by a sheet deployment structure.

According to a further feature of the present invention, the sheet of material is implemented as a continuous loop rotating between two conveyer rollers such that one surface of the sheet passes through a photic zone of the body of water flowing downstream.

According to a further feature of the present invention, there is also provided retrieving the at least one sheet of material and attached microbial biomass from the in-situ body of water.

According to a further feature of the present invention, the retrieving the sheet of material and attached microbial biomass from the in-situ body of water includes winding each of the sheets into a roll held by the sheet deployment structure.

According to a further feature of the present invention, there is also provided harvesting the microbial biomass from the sheets.

According to a further feature of the present invention, the harvesting the microbial biomass from the sheets includes charging the sheet of material and the attached microbial biomass into an anaerobic reactor to gasify the microbial biomass.

There is also provided according to the teachings of the present invention, a method for growing microbial communities in a body of water flowing downstream, which comprises: (a) inserting a free-floating bodies having a plurality of surfaces into an in-situ body of water flowing downstream; and (b) retaining the free-floating bodies in a portion of the in-situ body of water flowing downstream by way of a retaining element, thereby facilitating bio-remediating the in-situ body of water flowing downstream by way of microbial colonization on the surfaces.

According to a further feature of the present invention, there is also provided removing the free-floating bodies and attached microbial biomass from the in-situ body of water flowing downstream.

According to a further feature of the present invention, there is also provided harvesting the attached microbial biomass from the free-floating bodies.

According to a further feature of the present invention, the harvesting the attached microbial biomass from the free-floating bodies includes charging the free-floating bodies and attached microbial biomass into an anaerobic reactor to gasify the microbial biomass.

There is also provided according to the teachings of the present invention, a microbial growth sheet arrangement for supporting sheets of material for microbial growth in an in-situ body of water flowing downstream comprising: (a) at least one sheet, at least partially disposed in an in-situ body of water flowing downstream; and (b) a sheet deployment structure supporting each of the at least one sheet in the in-situ body of water flowing downstream so as to maximize contact between the body of water flowing downstream and surfaces of the sheets, thereby facilitating bio-remediation of the body of water by way of microbial colonization on the surfaces.

According to a further feature of the present invention, the sheet deployment structure includes at least one pivotally mounted roller, each of the at least one roller corresponding to each of the at least one sheet to facilitate unwinding of the sheets from a roll of the sheet held by the roller during deployment or winding of the sheet into a roll of the sheet held by the roller during retrieval.

According to a further feature of the present invention, the at least one pivotally mounted roller is driven by a powered drive mechanism.

According to a further feature of the present invention, the at least one sheet is implemented as a continuous loop rotating between two conveyer rollers such that one surface of the sheet passes through a photic zone of the body of water flowing downstream.

According to a further feature of the present invention, the at least one of the two conveyor rollers is driven by a powered drive mechanism.

### BRIEF DESCRIPTION OF THE DRAWINGS

The invention is herein described, by way of example only, with reference to the accompanying drawings, wherein:

FIG. 1 is a schematic representation of a series of microbial support structures implemented as growth sheets disposed in a flowing body of water.

FIG. 2 is a schematic representation of the structure of FIG. 1 with a flow baffle to generate mixing of flow layers of the water.

FIG. 3 is a schematic, side view of a microbial growth sheet depicting different growth zones.

FIG. 4 is a schematic, end view of a horizontal-roller growth-sheet support.

FIG. 5 and FIG. 6 are schematic side of a standing-roller, growth-sheet support structure, respectively.

FIG. 7 is a schematic, side view of a sheet conveyer arrangement for conveying the sheets while disposed vertically in a body of water to temporally expose bio-films to light.

FIG. 8 is a schematic, side view of a sheet conveyer arrangement for conveying the sheets while disposed horizontally in a body of water to temporally expose bio-films to light.

FIG. 9 is a schematic, top view of microbial growth structures implemented as free-floating, microbial support structures deployed in a flowing body of water.

FIG. 10 is a schematic, top view of a river diverted to improve eco-physiological properties facilitating microbial growth.

FIG. 11 is a flow chart depicting the steps involved in bio-remediating a contaminated body of flowing water and harvesting the resulting biomass.

### DESCRIPTION OF THE PREFERRED EMBODIMENTS

The present invention is a method for facilitating growth of microbial communities for in-situ bodies of flowing water for the sake of bio-remediating contaminated water and/or producing useful biomass.

Specifically, the present invention teaches introducing structures having large surface/volume ratios into bodies of flowing water in their natural setting for growing communities of diverse metabolic capacities capable of absorbing, metabolizing, modifying, detoxifying and quarantining pollutants.

The invention is relatively simple, low-cost technology exploiting natural processes without introducing mutant or new species into the environment and hence preserves the ecological homeostasis of the water body.

The principles and operation of the method according to the present invention may be better understood with reference to the drawings and the accompanying description.

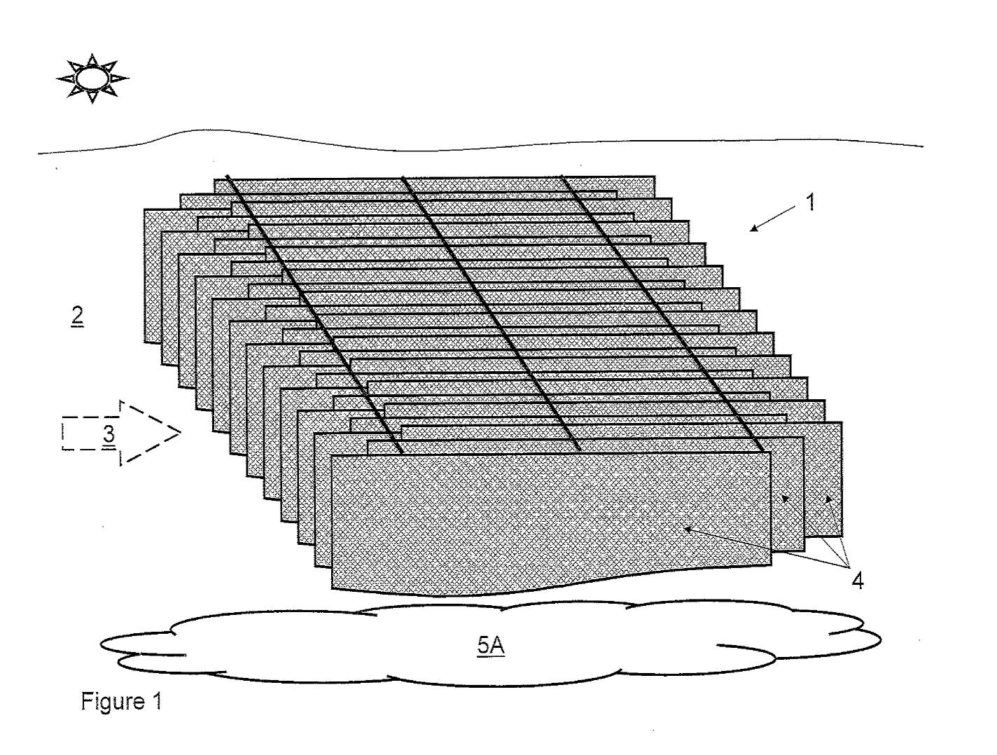

Turning now to the drawings, FIG. 1 depicts an exemplary, non-limiting embodiment of a microbial growth system 1 including growth structure 4 disposed in a substantially vertical manner in river 2, substantially parallel to the flow of current as designated by arrows 3. Growth structures 4 are implemented as sheets to provide maximum surface on which microbial colonies attach and form biofilms or biomats in a non-limiting exemplary embodiment. The microbial colonies metabolize pollutants absorbed from water flowing over growth sheets 4. It should be appreciated that configurations in which sheets 4 are disposed so as to form a serendipitous flow of water and/or are disposed non-vertically in river 2 are also included within the scope of the invention. It is advantageous as this point to define terms to be used throughout this document:
“Body of flowing water” refers to any body of water in motion like rivers, streams, oceans, underground bodies of flowing water, and springs. It should be appreciated that for the sake of convenience this document describes the present invention in the context of a river; however, all types of bodies of in-situ flowing water are included within the scope of the present invention.
“In-situ” refers to the location or the means generating the flow of the body of water. Specifically, “in-situ” means that the body of water is in its natural habitat. Man-made waterways and bodies of water formed by dams are also considered to be “in-situ” for the purposes of this document since man-made waterways possess flow dynamics and environmental factors found in totally natural settings. “In-situ” excludes bodies of water totally removed from their environment like those found in tanks or isolated mixing pools in which the water flow is generated by mechanical means.
“Downstream” refers to the direction of gravity flow of water including flow through man made waterways.
“Microbial colonies” include, but are not limited to biofilm forming Diatoms, Cyanobacteria, Chloroflexus-like bacteria, Colorless sulfur bacteria, green (non)sulfur bacteria, purple (non)sulfur bacteria, heterotrophs, chemotrophs, autotrophs, Eukaryotes, bacteria, Archaea, and viruses.
“Photic zone” refers to the depth from the water surface at which light intensity drops to 1% of its intensity at the surface.
   In an exemplary, non-limiting embodiment, growth sheets 4 are constructed from mesh polycarbonate screens having mesh sizes ranging from 1-2 millimeters or flexible plastic sheets. The light weight, biologically inert, and low cost make polycarbonate and plastics ideal candidates for growth sheets 4; however, it should be appreciated that any material having these characteristics are included within the scope of the present invention.

Sheets 4 are inserted into a body of water flowing downstream and held at a space of at least two centimeters apart to reduce shade emanating from neighboring sheets 4 that could retard desired microbial growth on both sides of each sheet 4. Sheet heights are chosen so that when the top of sheet 4 is disposed just below the water surface, the bottom portion extends to a depth exceeding the photic zone to provide growth surface for microbial organisms having metabolic capabilities in minimal light conditions thereby increasing the range of bioremediation possibilities. It should be appreciated that environments having a particular pollutant, the growth sheets and their configuration are tailored to enhance the particular microbial growth most effective at assimilating the target pollutant. Turbidity, flow speed, pollution type and quantity, sedimentation rate, water clarity, available light and natural predation rate are environmental variables defining the optimal number of growth sheet 4, their dimensions and configuration. An exemplary, non-limiting embodiment, a sheet arrangement in an illuminated, low speed, river of five meters in width, one meter in depth with a 30 centimeter photic zone would be constructed as follows: Fifty sheets of polycarbonate mesh screens one half meter in height and 100 meters in length spaced ten centimeters apart from each other are disposed lengthwise in the river. This configuration provides the following growth surface contacting polluted water as it flows between the sheets:

Exploited Sheet Surface Area:

100 meter length×0.3 photic zone depth×2 side×50 sheets=3000 m²

Water Volume (Assuming the River has Parallelepiped Geometry:

100 meter length×5 meter width×1 meter depth=500 m³

Surface/Volume=3000/500=6.0 m⁻¹

### Comparing to Natural Surface/Volume Ratio:

Volume remains unchanged: 500 m³
Surface area provided by two sides of the river also having a photic zone extending to a depth of 0.30 meter

100 meter length×0.3 meters photic zone×2 sides of the river=60 m².

The natural exploited Surface/Volume ratio is 60 m²/500 m³=0.12 m⁻¹.
Naturally occurring S/V=0.12 m⁻¹
Augmented S/V=6.0 m⁻¹

As shown, the present invention advantageously provides an improvement in surface/volume ratio by two orders of magnitude.

It should be appreciated that any growth structure arrangement is dependent on a large number of factors like pollution type and concentration, light intensity and wavelength, water temperature, turbidity, as noted above. Therefore, practitioners skilled in the art, capable of weighing the factors are able of designing the most effective sheet arrangement for each location.

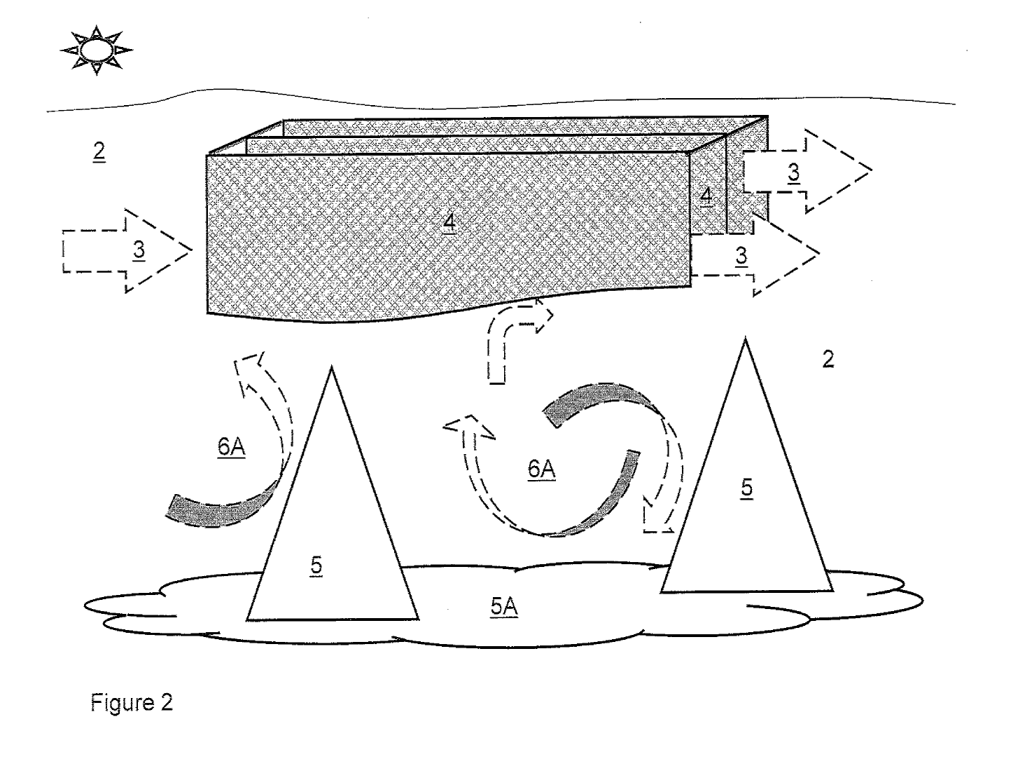

FIG. 2 depicts an exemplary, non-limiting embodiment of the microbial growth system 1 and flow baffles 5 spaced along river bed 5A. Flow baffles 5 create turbulence 6A thereby mixing stratified flow layers so that any pollutant flowing in lower layers also contacts microbial colonies found on the upper portion of growth sheets 4. Continuing with the above example, the river of five meters in width described above, flow baffles 5 of 25 centimeters in height are disposed every 2-5 meters.

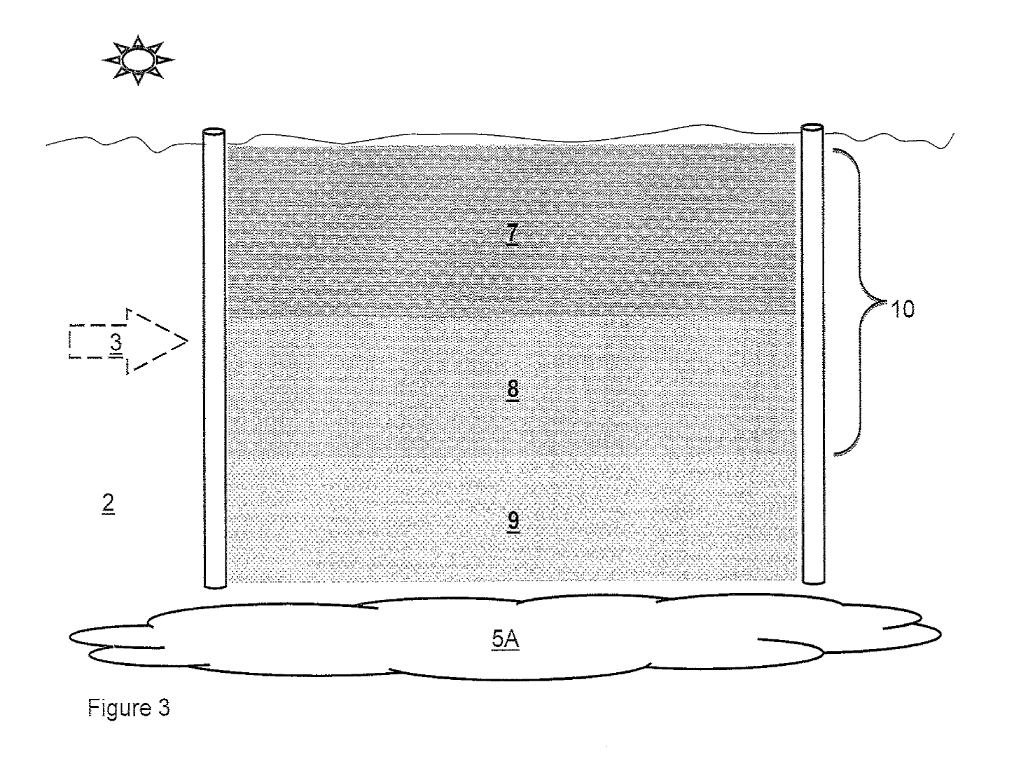

FIG. 3 depicts three different zones of complex microbial community that develop in accordance with the light attenuation gradient; two zones 7 8 are within the photic zone 10, and bottom zone 9 below the photic zone 10. Phototrophic organisms requiring abundant light, like Diatoms, some Cyanobacteria and Chloroflexus-like bacteria as well as non-phototrophic microorganisms like Heterotrophs colonize in zone 7 whereas organisms requiring lesser light like low-light species of Cyanobacteria and Heterotrophs, green (non)sulfur bacteria, purple (non)sulfur bacteria, low-light and some chemotrophs also colonize in zone 8. Heterotrophs and chemotrophs can grow below photic zone 10. It should be noted that microbial mat is three-dimensional and therefore has an additional photic zone typically extending 1-10 millimeters into the depth of the mat. In terms of the various zones extending into the depth of the mats, Heterotrophs also grow, whereas Phototrophs grow in light abundant or low light zones. As noted above, the present invention provides growth surfaces for all types of microbial colonization to facilitate growth of a diverse microbial community having a large range of bioremediation capabilities. Further detail regarding the effectiveness of microbial mats in bioremediation is found in U.S. Pat. No. 6,033,559 which is included in the present document in its entirety by reference.

There is a need to differentiate between biodegradable and non-biodegradable pollutants being assimilated by the microbial colonies. Biodegradable pollutants are converted into biocompatible matter and therefore may be allowed to be re-absorbed into the water. Under certain conditions microbial growth is removed from growth sheets 4 by way of fish, snails, ducks and other organisms inhabiting the river. However, non-biodegradable materials, like metal-based pollutants, must be removed from the system to avoid being reintroduced into the environment upon microbial cell death or predation.

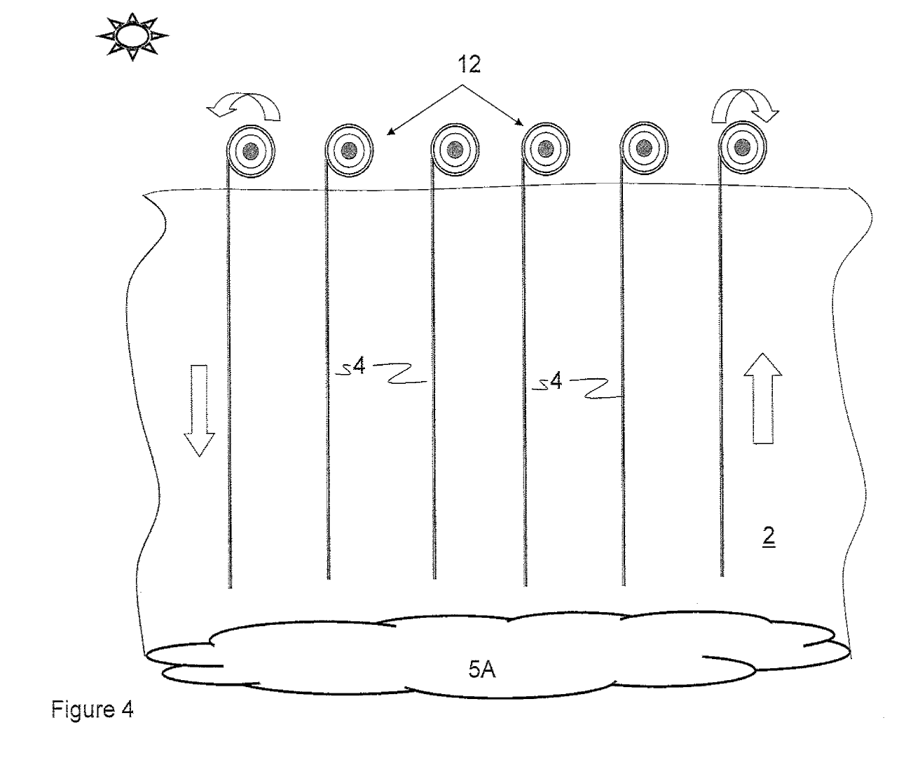

FIG. 4 is an end view, looking downstream, of a series of rollers 12 from which growth sheets 4 are unwound into river 2 upon deployment to a depth extending slightly beyond the photic zone in exemplary, non-limiting embodiments. The sheets are weighted or anchored to ensure that maximum surface area contacts the water flowing downstream if flow rate and turbidity necessitates. Upon sheet retrieval, sheets 4 are rewound into rolls by way of rollers 12 to remove attached microbial colonies for the sake of harvesting and/or non-biodegradable pollutants from the environment. In a preferred, non-limiting embodiment, rollers 12 are rollably mounted on a sheet deployment structure, generally designated 15, and anchored to the river banks or any other geographical or man-made feature capable of providing stability. Sheets 4 are wound up manually by way of a crank and gear assembly in locations lacking the technological infrastructure; however, sheet deployment structures powered by flowing water, wind, spring arrangements, electric motors, or combustion engines are included within the scope of the present invention. After sheets 4 and its content are wound up into a roll, the accumulated biomass is then harvested from sheets 4 and used for a variety of applications as will be discussed.

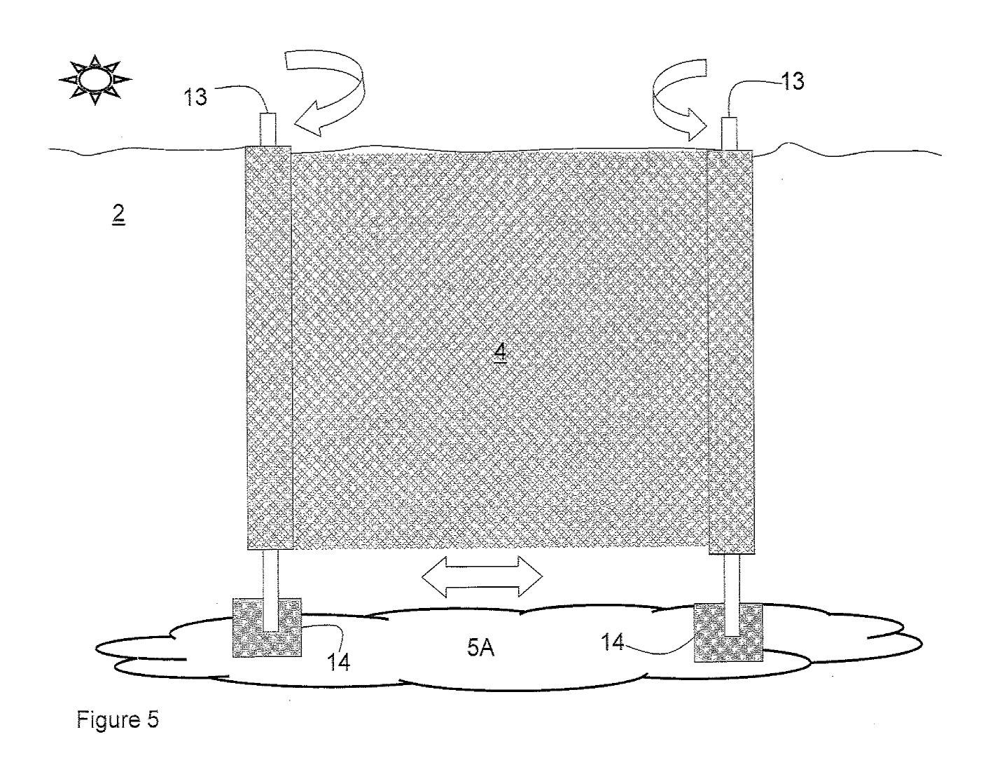

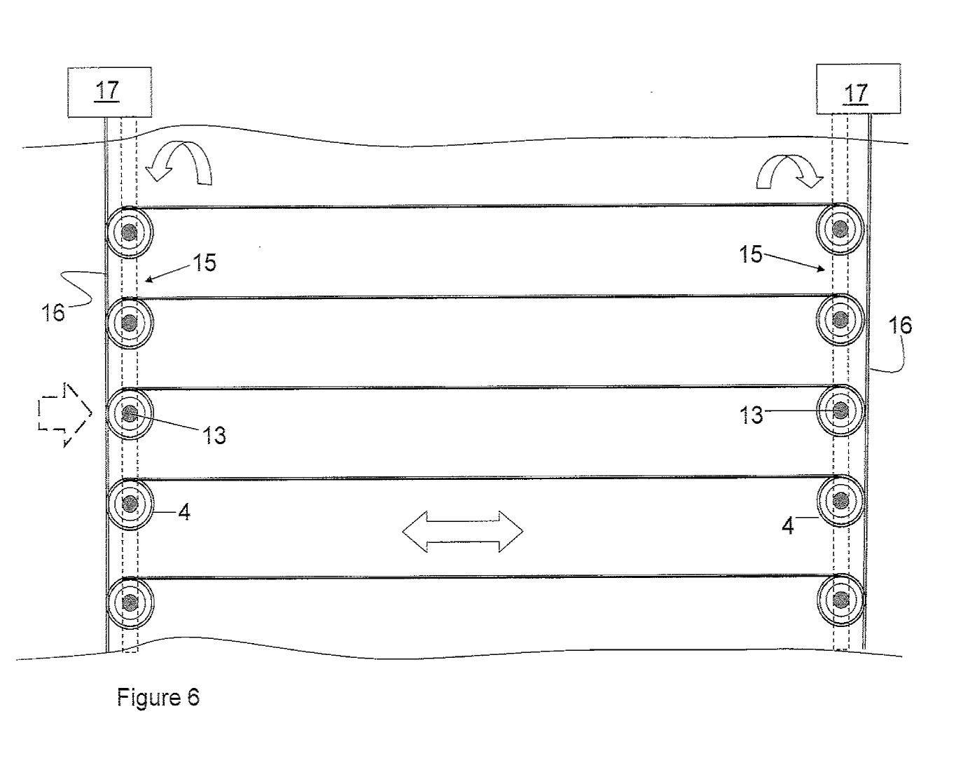

FIGS. 5 and 6 are side and top views, respectively of a non-limiting, alternative embodiment of sheet deployment structure 15. Sheet 4 is rolled up by way of roller 13 vertically mounted in roller mounts 14 disposed at separate points in river bed 5A. Rollers 13 are supported by a roller support 15A as shown in FIG. 6 and rotated by way of rotator 17 linked to rollers by way of worm gear 16 in a non-limiting, exemplary embodiment. Rotator 17 is powered by any of the mechanisms discussed above. During deployment a user connects each sheet 4 to a corresponding downstream (or upstream) roller 13 or to an anchor structure (not shown) so that each sheet 4 spans a portion of the river length so as to maximize surface contact with the flowing water and unwinds each rolled sheet 4 held by each corresponding roller 13 as depicted in FIG. 6. After deployment, sheets 4 are held in their deployment position until sufficient biomass has accumulated on sheets 4 to justify harvesting or there exists a danger of assimilated non-biodegradable pollutants being reabsorbed into the environment. The time between deployment and retrieval depends on a wide variety of conditions as noted above, and therefore requires the judgment of one skilled in the art. During retrieval, sheets 4 and the attached biomass are rolled up onto rollers 13 either by first releasing sheets 4 from their anchor structures and re-rolling them onto their original rollers 13 or rolling them onto an opposing roller 13 as depicted in FIGS. 5 and 6. After sheets 4 and the attached biomass are rolled up, the rolled sheets are either removed from rollers 13 or roller 13 in its entirety together with rolled sheets 4 are removed from sheet deployment structure 15. The attached biomass is harvested from the sheet 4 as will be discussed. Embodiments in which rollers 13 are removed from deployment structure, a clean roll of sheet material 4 is inserted into deployment structure in preparation for subsequent deployment and bioremediation.

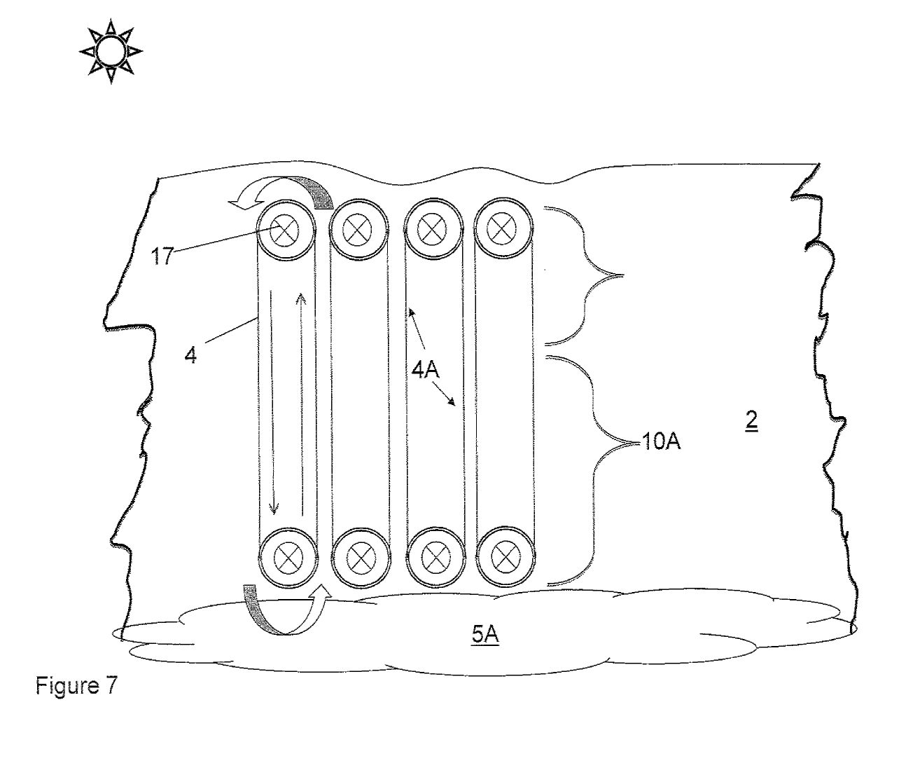

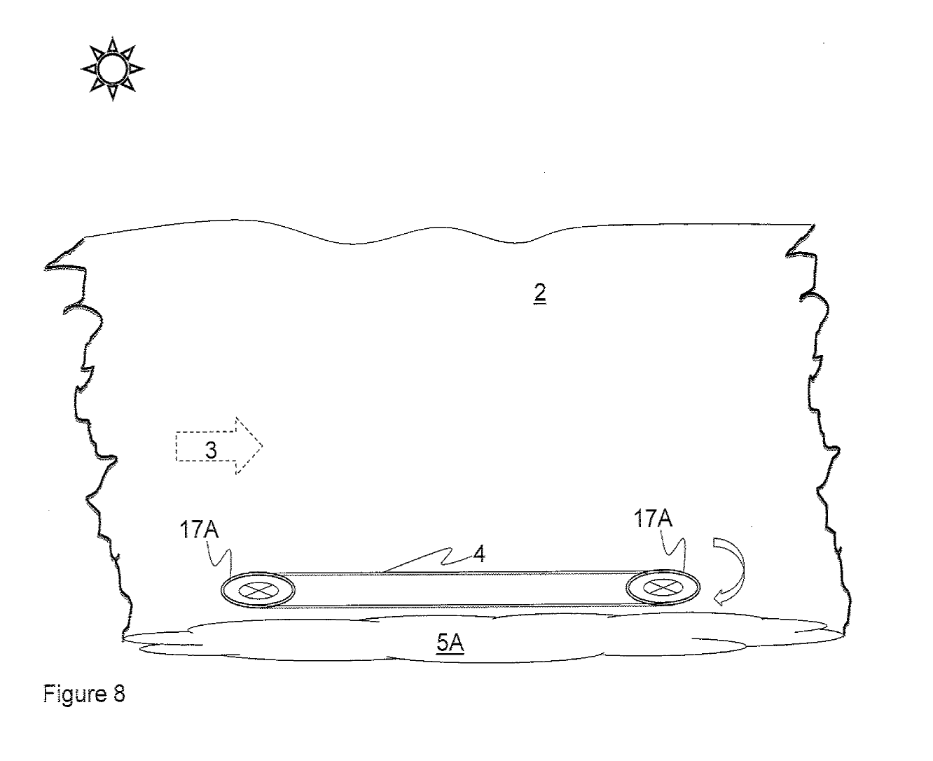

FIG. 7 depicts non-limiting, alternative embodiment directed at augmenting phototrophic microbial growth by exposing the entire outer surface of sheet to light while preserving a non-phototrophic zone on the inner surface 4A to advantageously provide suitable light conditions for both non-phototrophic and phototrophic microbial growth to provide a wide range of metabolic capabilities. Sheets 4 are implemented as a continuous loop rotating vertically between conveyer rollers 17. Microbial colonies attached to the outer surface of sheets 4 are temporally exposed to abundant and low light conditions as they pass through photic and non non-photic zones 10 and 10A. Non-phototropic microbial colonies attached to the inner surface 4A are maintained in a state of relative darkness. Conveyer rollers 17 are powered as noted above. This embodiment also provides for the convenient removal of sheets 4 by winding them up on one of the conveyer rollers 17 after removing sheet 4 from one conveyor roller 17. It should be appreciated that embodiments in which the conveyer arrangement is integral or non-integral to sheet deployment structure 15 are included within the scope of the present invention. It should be noted that sheets 4 disposed in non-perpendicular angles to a horizontal plate are also included within the scope of the present invention as will be further discussed.

In reference to FIG. 5, shallow streams requiring sheets 4 to be disposed in a substantially horizontal position, sheet 4 is also implemented as a continuous loop rotated between conveyor rollers 17A to achieve temporal exposure of all microbial mats having developed on the outer surface to abundant and low light conditions while bio-films formed on the inner surface 4A remain relatively in a non-photic zone as described above. It should be noted that variant embodiments in which sheet 4 is spread in a substantially horizontal plane slightly above riverbed 5A and supported by way of a shallow water deployment structure (not shown) is included within the scope of the present invention. An exemplary, non-limiting embodiment of the shallow water deployment structure is implemented as a series of inverted “U” shaped frames disposed in the middle of river 2. The frames have a height less than the depth of river 2 and serve as a backbone for sheet 4 spread over them and anchored along the edges of river 2. Such variant embodiments facilitate the growth of a phototrophic mat on the upper surface and a non-phototrophic mat on the bottom surface. It should be appreciated that all shallow water deployment structures providing stability of sheets 4 spread in shallow water are included within the scope of the invention.

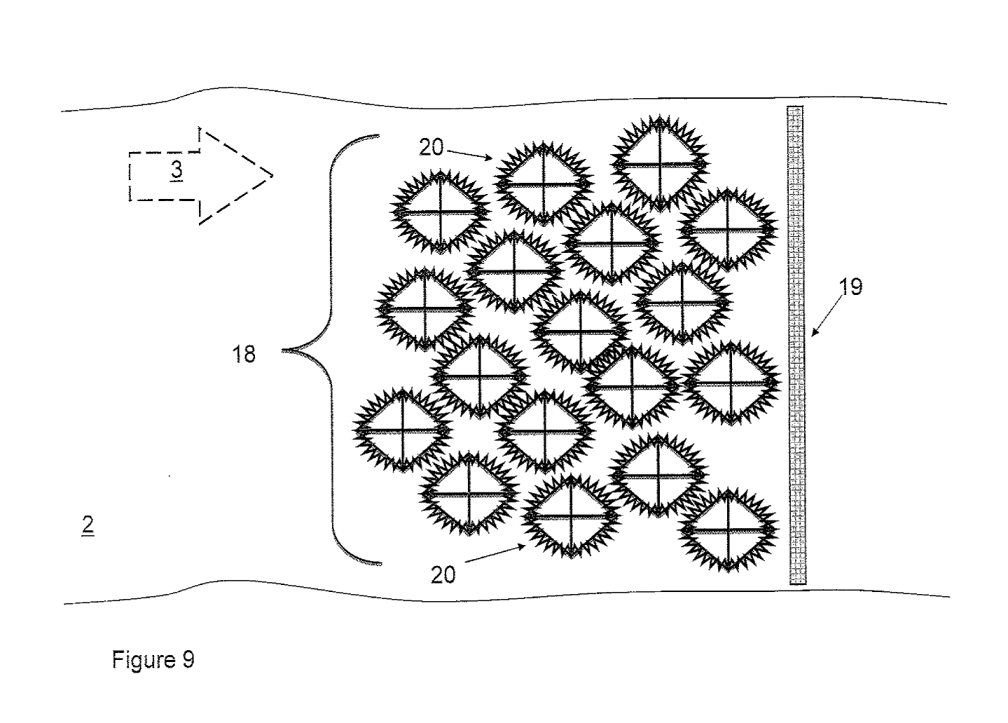

FIG. 9 depicts a non-limiting, alternative embodiment of free-floating, growth structures 18 having a multiplicity of growth surfaces 20. Growth structures are contained in a suitable area within the body of flowing water by way of retaining element 19 capable of allowing water to flow downstream while containing growth structures 18. Containing element 19 is implemented as a screen in an exemplary non-limiting embodiment; however it should be appreciated that other structures having the above functionality are included within the scope of the present invention. After free-floating growth structures 18 have been retained for time sufficient for the development of microbial colonization and bioremediation, free-floating growth structures 20 are removed from river 2 in preparation for harvesting. Floating growth structures 18 are constructed in a non-limiting preferred embodiment from low density polymeric materials to enable flotation. It should be appreciated, however, that all materials providing flotation at any height in the river are included within the scope of the present invention. Furthermore, all suspension configurations or, alternatively, anchoring configurations of floating growth surfaces are also included within the scope of the present invention.

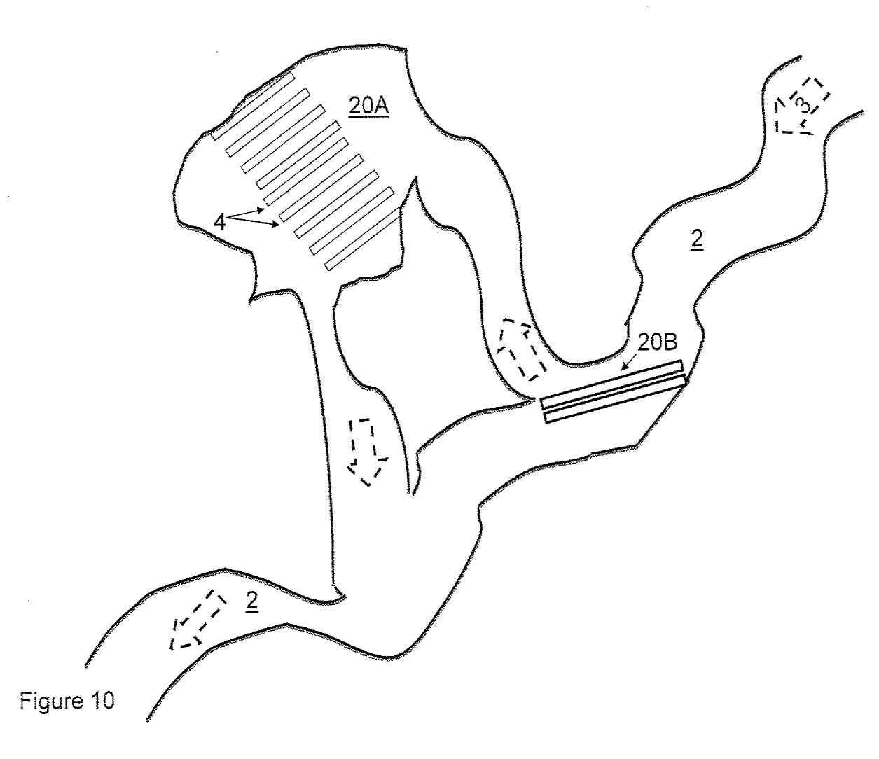

FIG. 10 depicts an in-situ water flow arrangement in which the natural flow of a river has been diverted for the sake of gaining improved eco-physiological properties. Typically, such rivers have clear water in which the photic zone extends to the riverbed. The river 2 is diverted by way of gate 20B and passes through a man-made, deep pool in which a series of growth sheets 4 are disposed to facilitate growth of non-phototrophic micro-organisms in addition to phototrophic micro-organisms that thrive in the photic zone. After passing through sheets 4, the river, returns to its natural path. As noted above, manmade structures in which the water flows by way of gravity are considered in-situ for the purposes of this document.

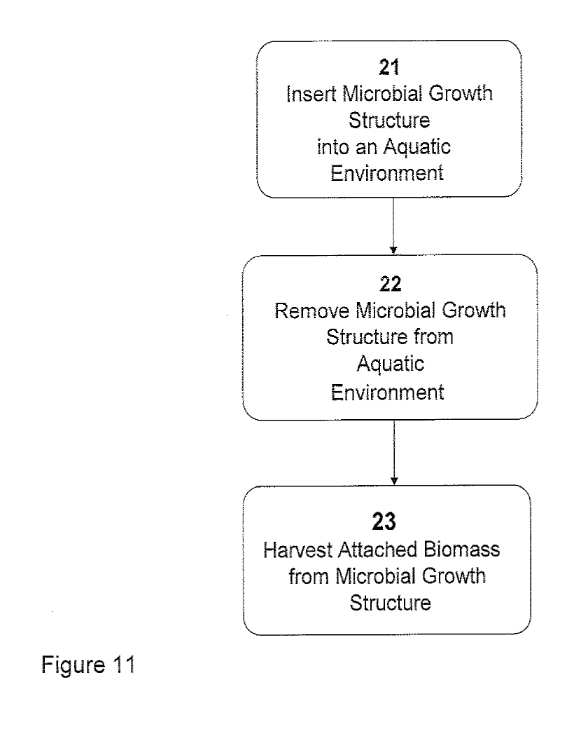

FIG. 11 depicts a three step process involved in the present invention; inserting microbial growth structures into an aquatic environment 21, removing the growths structures together with any accumulated microbial growth 22, and harvesting the attached biomass 23. Harvesting, in a non-limiting, exemplary embodiment, is executed by charging the growth structures together with the attached biomass into an anaerobic reactor to produce methane, hydrogen and other materials as is known to those skilled in the art. The growth surfaces are thereby cleaned and can then be re-inserted into the river. Alternatively, the biomass is harvested by chipping off the microbial growth or by other removal techniques known to those skilled in the art.

The biomass applications can be used as fish food, organic fertilizer, and/or may be bio-mined to recover rare elements extracted from the assimilated pollutants or by-products of metabolized pollutants.

The present invention is also useful for bio-remediating contaminated aquatic environments containing specific or complex chemical pollutants including fertilizers, pesticides metal and inorganic contaminates.

It will be appreciated that the above descriptions are intended only to serve as examples, and that many other embodiments are possible within the scope of the present invention as defined in the appended claims.

What is claimed is:

1. A method for growing microbial communities in flowing water, which comprises:
a) Inserting at least a portion of at least one sheet of material into an in-situ body of water flowing downstream; and
b) holding said sheet in said in-situ body of water flowing downstream by way of a sheet deployment structure configured to hold said sheet so as to maximize contact of the body of water flowing downstream with surfaces of said sheets, thereby facilitating bio-remediation of the body of water by way of microbial colonization on the surfaces of said at least one sheet.

2. The method of claim 1 wherein said sheet of material includes a screen.

3. The method of claim 1, wherein said sheet of material includes a polymeric material.

4. The method of claim 1, wherein said inserting at least one sheet of material includes orientating said sheet in a substantially non-vertical plane.

5. The method of claim 1 wherein said inserting at least one sheet of material includes orientating said sheet of material in a substantially vertical plane.

6. The method of claim 5, wherein said inserting at least one sheet of material includes inserting each of at least one of said sheets into the in-situ body of water by unwinding each of said at least one sheet from a corresponding rolled sheet of material held by a sheet deployment structure.

7. The method of claim 1, wherein said sheet of material is implemented as a continuous loop rotating between two conveyer rollers such that one surface of said sheet passes through a photic zone of the body of water flowing downstream.

8. The method of claim 1, which further comprises retrieving said at least one sheet of material and attached microbial biomass from the in-situ body of water.

9. The method of claim 8 wherein retrieving said sheet of material and attached microbial biomass from the in-situ body of water includes winding each of said sheets into a roll held by said sheet deployment structure.

10. The method of claim 8, which further comprises harvesting the microbial biomass from said sheets.

11. The method of claim 10, wherein said harvesting the microbial biomass from said sheets includes charging said sheet of material and the attached microbial biomass into an anaerobic reactor to gasify the microbial biomass.

12. A method for growing microbial communities in a body of water flowing downstream, which comprises:
(a) inserting a free-floating bodies having a plurality of surfaces into an in-situ body of water flowing downstream; and
(b) retaining said free-floating bodies in a portion of the in-situ body of water flowing downstream by way of a retaining element, thereby facilitating bio-remediating the in-situ body of water flowing downstream by way of microbial colonization on said surfaces.

13. The method of claim 12, which further comprises removing said free-floating bodies and attached microbial biomass from the in-situ body of water flowing downstream.

14. The method of claim 13, which further comprises harvesting the attached microbial biomass from said free-floating bodies.

15. The method of claim 14, wherein said harvesting the attached microbial biomass from said free-floating bodies includes charging said free-floating bodies and attached microbial biomass into an anaerobic reactor to gasify the microbial biomass.

16. A microbial growth sheet arrangement for supporting sheets of material for microbial growth in an in-situ body of water flowing downstream comprising:
(a) at least one sheet, at least partially disposed in an in-situ body of water flowing downstream; and
(b) a sheet deployment structure supporting each of said at least one sheet in the in-situ body of water flowing downstream so as to maximize contact between the body of water flowing downstream and surfaces of said sheets, thereby facilitating bio-remediation of the body of water by way of microbial colonization on said surfaces.

17. The microbial growth sheet arrangement of claim 16, wherein sheet deployment structure includes at least one pivotally mounted roller, each of said at least one roller corresponding to each of said at least one sheet to facilitate unwinding of said sheets from a roll of said sheet held by said roller during deployment or winding of said sheet into a roll of said sheet held by said roller during retrieval.

18. The microbial growth sheet arrangement of claim 17, wherein said at least one pivotally mounted roller is driven by a powered drive mechanism.

19. The microbial growth sheet arrangement of claim 16, wherein said at least one sheet is implemented as a continuous loop rotating between two conveyer rollers such that one surface of said sheet passes through a photic zone of the body of water flowing downstream.

20. The microbial growth sheet arrangement of claim 19, wherein at least one of said two conveyor rollers is driven by a powered drive mechanism.
